(19) United States
(12) Patent Application Publication
(10) Pub. No.: US 2011/0301478 A1
(43) Pub. Date: Dec. 8, 2011

(54) MANAGEMENT OF MEDICAL INFORMATION

(75) Inventor: Tomer Ben-Sira, Tel-Aviv (IL)

(73) Assignee: McKesson Financial Holdings Limited

(21) Appl. No.: 12/795,029

(22) Filed: Jun. 7, 2010

(51) Int. Cl. A61B 5/0452; G06F 15/16; G06Q 50/00

(52) U.S. Cl. 600/515; 705/2; 709/204

(57) ABSTRACT

Systems, methods, apparatus, and computer program products are provided for managing medical information. For example, in one embodiment, medical information may be transmitted from a device such as an electrocardiographic device to a management system. The management system can transmit at least a portion of the medical information to a mobile device associated with, for example, a medical provider for remote diagnosis. In response to receiving a particular diagnosis, the management system may automatically initiate one or more protocols.

### BACKGROUND

Certain medical conditions require immediate treatment by medical providers. For example, a patient suffering from an ST segment elevation myocardial infarction may need to have a coronary angioplasty performed within 90 minutes. Thus, a need exists for assisting medical providers in promptly evaluating and diagnosing medical conditions regardless of their location.

### BRIEF SUMMARY

In general, embodiments of the present invention provide systems, methods, apparatus, and computer program products for managing medical information.

In one aspect, a method for managing electrocardiographic information is provided. In one embodiment, the method comprises (1) receiving electrocardiographic information associated with a patient; (2) electronically identifying a provider associated with a health care facility; and (3) transmitting at least a portion of the electrocardiographic information to a mobile device associated with the provider. The method may also comprise (4) in response to transmitting at least a portion of the electrocardiographic information to the mobile device associated with the provider, receiving a patient status from the mobile device, wherein the patient status (a) is determined by the provider based at least in part on the at least a portion the electrocardiographic information and (b) is input by the provider via the mobile device; and (5) in response to receiving the patient status from the mobile device, automatically initiating a protocol associated with the patient status.

In accordance with yet another aspect, a computer program product for managing electrocardiographic information is provided. The computer program product may comprise at least one computer-readable storage medium having computer-readable program code portions stored therein, the computer-readable program code portions comprising executable portions configured to (1) receive electrocardiographic information associated with a patient; (2) identify a provider associated with a health care facility; and (3) transmit at least a portion of the electrocardiographic information to a mobile device associated with the provider. The computer-readable program code portions may also comprise executable portions configured to (4) in response to transmitting at least a portion of the electrocardiographic information to the mobile device associated with the provider, receive a patient status from the mobile device, wherein the patient status (a) is determined by the provider based at least in part on the at least a portion the electrocardiographic information and (b) is input by the provider via the mobile device; and (5) in response to receiving the patient status from the mobile device, automatically initiate a protocol associated with the patient status.

In accordance with yet another aspect, an apparatus comprising at least one processor and at least one memory including computer program code is provided. In one embodiment, the at least one memory and the computer program code may be configured to, with the processor, cause the apparatus to at least (1) receive electrocardiographic information associated with a patient; (2) identify a provider associated with a health care facility; and (3) transmit at least a portion of the electrocardiographic information to a mobile device associated with the provider. The at least one memory and the computer program code may also be configured to, with the processor, cause the apparatus to at least (4) in response to transmitting at least a portion of the electrocardiographic information to the mobile device associated with the provider, receive a patient status from the mobile device, wherein the patient status (a) is determined by the provider based at least in part on the at least a portion the electrocardiographic information and (b) is input by the provider via the mobile device; and (5) in response to receiving the patient status from the mobile device, automatically initiate a protocol associated with the patient status.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is an overview of system that can be used to practice various embodiments of the present invention.

FIG. 2 is an exemplary schematic diagram of a management system according to one embodiment of the present invention.

FIG. 3 is an exemplary schematic diagram of a mobile device according to one embodiment of the present invention.

FIGS. 4-5 are flowcharts illustrating operations and processes that can be used in accordance with various embodiments of the present invention.

FIGS. 6-7 are universal input and output produced by one embodiment of the invention.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. Like numbers refer to like elements throughout.

### I. Methods, Apparatus, Systems, and Computer Program Products

As should be appreciated, various embodiments may be implemented in various ways, including as methods, apparatus, systems, or computer program products. Accordingly, various embodiments may take the form of an entirely hardware embodiment or an embodiment in which a processor is programmed to perform certain steps. Furthermore, various implementations may take the form of a computer program product on a computer-readable storage medium having computer-readable program instructions embodied in the storage medium. Any suitable computer-readable storage medium may be utilized including hard disks, CD-ROMs, optical storage devices, or magnetic storage devices.

Various embodiments are described below with reference to block diagrams and flowchart illustrations of methods, apparatus, systems, and computer program products. It should be understood that each block of the block diagrams and flowchart illustrations, respectively, may be implemented in part by computer program instructions, e.g., as logical steps or operations executing on a processor in a computing system. These computer program instructions may be loaded onto a computer, such as a special purpose computer or other programmable data processing apparatus to produce a specifically-configured machine, such that the instructions which execute on the computer or other programmable data processing apparatus implement the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the functionality specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide operations for implementing the functions specified in the flowchart block or blocks.

Accordingly, blocks of the block diagrams and flowchart illustrations support various combinations for performing the specified functions, combinations of operations for performing the specified functions and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or operations, or combinations of special purpose hardware and computer instructions.

### II. Exemplary System Architecture

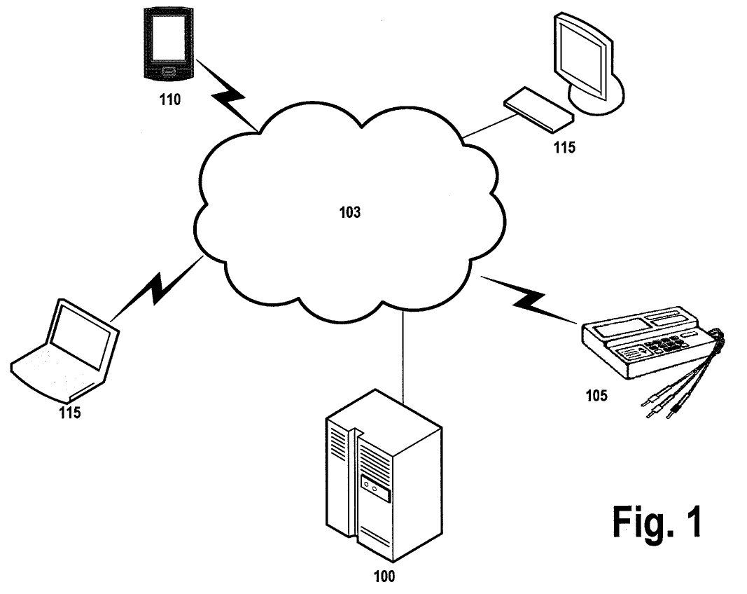

FIG. 1 provides an illustration of a system that can be used in conjunction with various embodiments of the present invention. As shown in FIG. 1, the system may include one or more management systems 100, one or more networks 103, one or more electrocardiogram (“ECG” or “EKG”) devices 105, one or more mobile devices 110, and one or more user computing devices 115. Each of the components of the system may be in electronic communication with, for example, one another over the same or different wireless or wired networks including, for example, a wired or wireless Personal Area Network (“PAN”), Local Area Network (“LAN”), Metropolitan Area Network (“MAN”), Wide Area Network (“WAN”), or the like. Additionally, while FIG. 1 illustrates certain system entities as separate, standalone entities, the various embodiments are not limited to this particular architecture.

### 1. Exemplary Management System

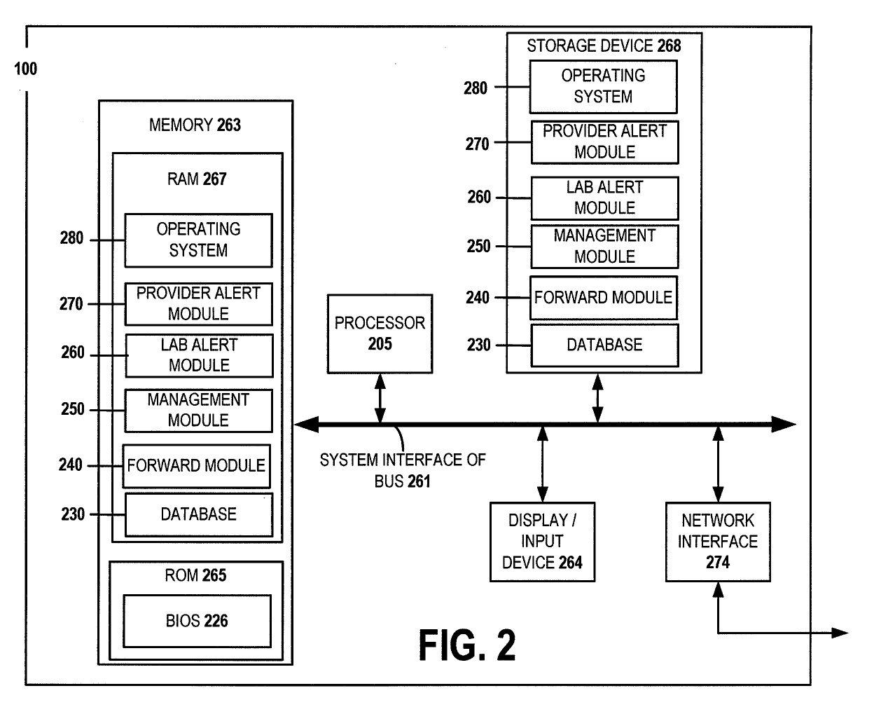

FIG. 2 provides a schematic of a management system 100 according to one embodiment of the present invention. In general, the term “management system” may refer to, for example, any computer, computing device, mobile phone, desktop, notebook or laptop, distributed system, server, blade, gateway, switch, or other processing device adapted to perform the functions described herein. As will be understood from this figure, in this embodiment, the management system 100 includes a processor 205 that communicates with other elements within the management system 100 via a system interface or bus 261. The processor 205 may be embodied in a number of different ways. For example, the processor 205 may be embodied as a processing element, a coprocessor, a controller or various other processing devices including integrated circuits such as, for example, an application specific integrated circuit (“ASIC”), a field programmable gate array (“FPGA”), a hardware accelerator, and/or the like.

In an exemplary embodiment, the processor 205 may be configured to execute instructions stored in the device memory or otherwise accessible to the processor 205. As such, whether configured by hardware or software methods, or by a combination thereof, the processor 205 may represent an entity capable of performing operations according to embodiments of the present invention while configured accordingly. A display device/input device 264 for receiving and displaying data may also be included in the management system 100. This display device/input device 264 may be, for example, a keyboard or pointing device that is used in combination with a monitor. The management system 100 further includes memory 263, which may include both read only memory (“ROM”) 265 and random access memory (“RAM”) 267. The management system's ROM 265 may be used to store a basic input/output system (“BIOS”) 226 containing the basic routines that help to transfer information to the different elements within the management system 100.

In addition, in one embodiment, the management system 100 includes at least one storage device 268, such as a hard disk drive, a CD drive, and/or an optical disk drive for storing information on various computer-readable media. The storage device(s) 268 and its associated computer-readable media may provide nonvolatile storage. The computer-readable media described above could be replaced by any other type of computer-readable media, such as embedded or removable multimedia memory cards (“MMCs”), secure digital (“SD”) memory cards, Memory Sticks, electrically erasable programmable read-only memory (“EEPROM”), flash memory, hard disk, or the like. Additionally, each of these storage devices 268 may be connected to the system bus 261 by an appropriate interface.

Furthermore, a number of program modules may be stored by the various storage devices 268 and/or within RAM 267. Such program modules may include an operating system 280, a provider alert module 270, a lab alert module 260, a management module 250, and a forward module 240. These modules may control certain aspects of the operation of the management system 100 with the assistance of the processor 205 and operating system 280—although their functionality need not be modularized. In addition to the program modules, the management system 100 may store or be connected to one or more databases (e.g., database 230) with one or more tables stored therein.

Also located within the management system 100, in one embodiment, is a network interface 274 for interfacing with various computing entities. This communication may be via the same or different wired or wireless networks (or a combination of wired and wireless networks), as discussed above. For instance, the communication may be executed using a wired data transmission protocol, such as fiber distributed data interface (“FDDI”), digital subscriber line (“DSL”), Ethernet, asynchronous transfer mode (“ATM”), frame relay, data over cable service interface specification (“DOCSIS”), or any other wired transmission protocol. Similarly, the management system 100 may be configured to communicate via wireless external communication networks using any of a variety of protocols, such as 802.11, general packet radio service (“GPRS”), wideband code division multiple access (“W-CDMA”), or any other wireless standard or protocol. Via the network interface 274, the management system 100 may be capable of sending and receiving messages to a variety of computing entities, such as the mobile device 110.

It will be appreciated that one or more of the management system's 100 components may be located remotely from other management system 100 components. Furthermore, one or more of the components may be combined and additional components performing functions described herein may be included in the management system 100.

### 2. Exemplary Electrocardiographic Device

In one embodiment, the ECG device 105 (e.g., ECG cart or defibrillator) may be configured to capture the electrical activity of a heart and output the activity as waveforms (e.g., P-QRS-T waves) or traces. For example, the ECG device 105 may use any number of leads, such as 3 leads, 5 leads, or 12 leads, to capture the electrical heart activity. The ECG device 105 can then output the electrical activity as waveforms/traces in a variety of ways, such as printing the waveforms/traces on graph paper, storing the waveforms/traces in various electronic formats, and/or transmitting the waveforms/traces to various computing entities. As will be recognized, the ECG device 105 may be, for example, affixed to a cart in a hospital or disposed in an ambulance or helicopter. Depending on the configuration of the ECG device 105, the ECG device 105 may include (or be in communication with) an antenna, a transmitter, a receiver, a network interface, and a processing device to allow the ECG device 105 to communicate information to and receive information from other computing entities.

### 3. Exemplary Mobile Device

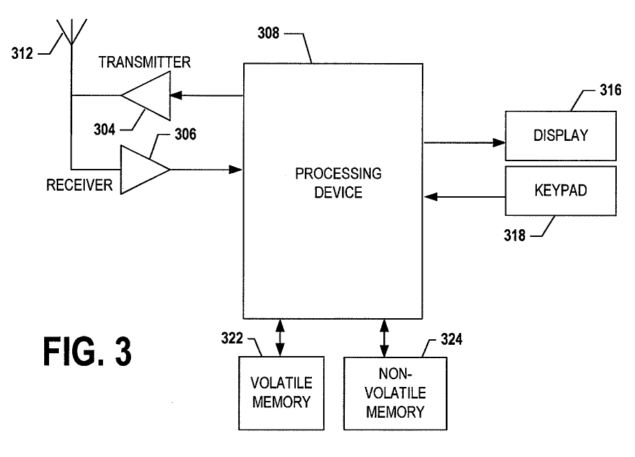

FIG. 3 provides an exemplary schematic representative of a mobile device 110 that can be used in conjunction with embodiments of the present invention. As shown in FIG. 3, the mobile device 110 may include an antenna 312, a transmitter 304, a receiver 306, a network interface 320, and a processing device 308 (e.g., a processor, controller, and/or the like) that provides signals to the transmitter 304 (and/or network interface 320) and receives signals from receiver 306 (and/or network interface 320).

The signals provided to the transmitter 304 (and/or network interface 320) and received from the receiver 306 (and/or network interface 320) may include signaling information in accordance with an air interface standard of applicable wireless systems. In this regard, the mobile device 110 may be capable of operating with one or more air interface standards, communication protocols, modulation types, and access types. More particularly, the mobile device 110 may operate in accordance with any of a number of second-generation (“2G”), third-generation (“3G”), and/or fourth-generation (“4G”) standards and protocols, and/or the like. Further, for example, the mobile device 110 may operate in accordance with any of a number of different wireless networking techniques, including Bluetooth, IEEE 802.11 (“Wi-Fi”), 802.16 (“WiMAX”), ultra wideband (“UWB”), and/or the like. Via these communication standards and protocols, the mobile device 110 can communicate with the management system 110, for example. The mobile device 110 can also download changes, add-ons, and updates, for instance, to its firmware, software (e.g., including modules), and operating system.

The mobile device 110 may also comprise a user interface (that can include a display 316 coupled to a processing device 308) and/or a user input interface (coupled to the processing device 308). The user input interface can comprise any of a number of devices allowing the mobile device 110 to receive input and/or data, such as a keypad 318, a touch display, voice or motion interfaces, or other input device such as a remote control. The mobile device 110 can also include volatile memory 322 and/or non-volatile memory 324, which can be embedded and/or may be removable. For example, the non-volatile memory may be embedded or removable multimedia memory cards (“MMCs”), secure digital (“SD”) memory cards, Memory Sticks, EEPROM, flash memory, hard disk, or the like. The memory can store any of a number of pieces or amount of information and data used by the mobile device 110 to implement the functions of the mobile device 110. The memory can also store content, such as program code for an application and/or other programs.

### 4. User Computing Devices

The user computing devices 115 may each include one or more components that are functionally similar to those of the management system 100 and/or mobile device 110. For example, in one embodiment, each user computing device 115 may include: (1) a processor that communicates with other elements via a system interface or bus; (2) a display device/input device; (3) memory including both ROM and RAM; (4) a storage device; and (5) a network interface. These architectures are provided for exemplary purposes only and are not limiting to the various embodiments. The term “computing device” is used generically to refer to any computer, mobile phone, computing device, desktop, notebook or laptop, distributed system, server, gateway, switch, or other processing device adapted to perform the functions described herein.

### III. Exemplary System Operation

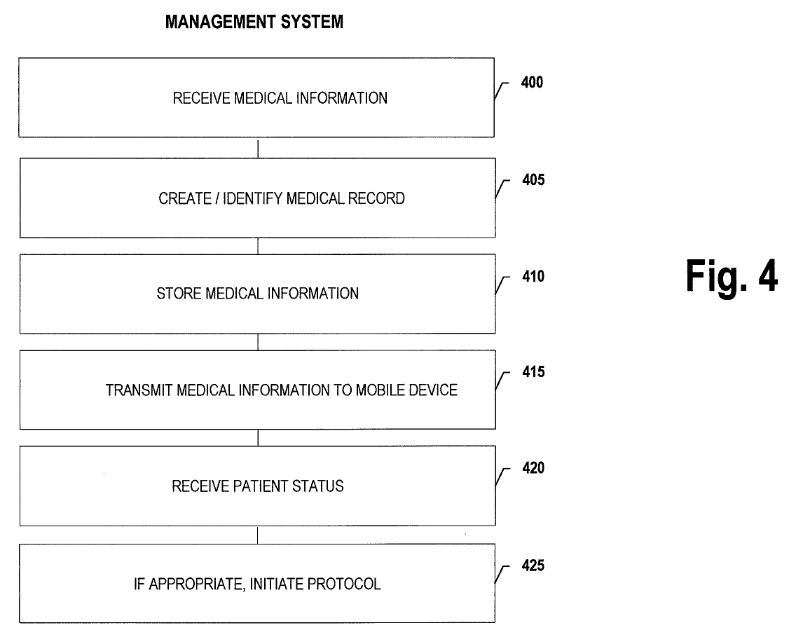

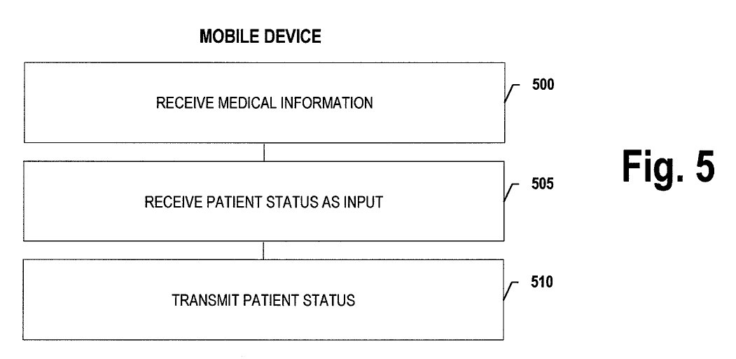

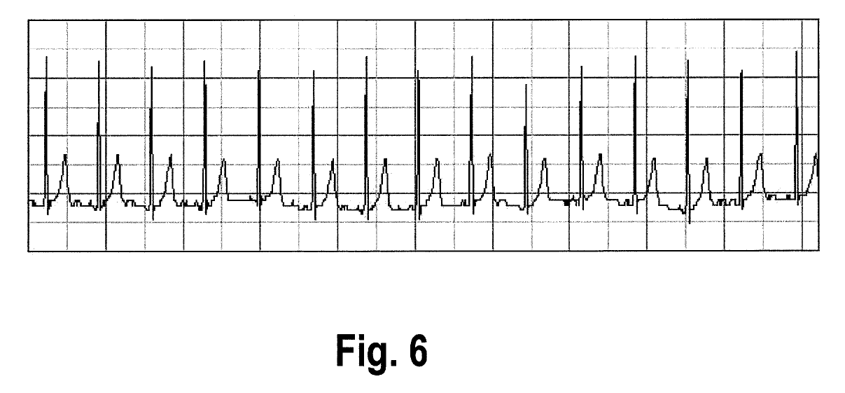

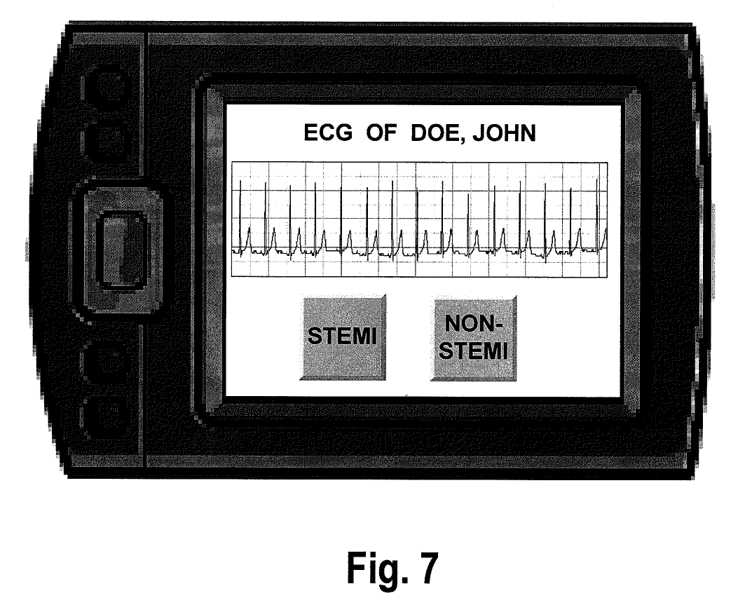

Reference will now be made to FIGS. 4-7. FIGS. 4-5 provide flowcharts illustrating operations that may be performed to assist medical providers in evaluating and diagnosing medical conditions. FIGS. 6-7 show universal input and output produced by one embodiment of the present invention.

To aid in understanding certain embodiments, the following sections describe embodiments of the present invention in the context of a patient believed to be suffering from an ST segment elevation myocardial infarction (“STEMI”). As will be recognized, though, embodiments of the present invention may be used to assist various types of medical providers in evaluating and diagnosing a variety of medical conditions.

a. Generation of Medical Information

In one embodiment, the process may begin with a patient suspected of suffering a medical condition, such as a STEMI. For example, medical providers (e.g., emergency response medical providers) may be dispatched to respond to a 911 call and attend to the patient at his home, business, church, and/or other location. In another embodiment, the patient may be attended to by medical providers (e.g., emergency room medical providers) in a health care facility, such as a hospital, a doctor's office, an urgent care center, and/or other facility. Regardless of location, in one embodiment, the patient may undergo a medical procedure, such as an electrocardiogram procedure. The electrocardiogram procedure may be performed by, for example, emergency response and/or emergency room medical providers with the aid of an ECG device 105. In one embodiment, the ECG device 105 may be a defibrillator transported to the patient's location, for example, in a rescue vehicle or an air ambulance. The rescue vehicle or air ambulance may also be used to transport the patient to a health care facility—i.e., at times the ECG device 105 may be remote from a health care facility. In another embodiment, the ECG device 105 may be disposed on an ECG cart for use within a health care facility. As will be recognized, a variety of ECG devices 105 (and/or other medical devices) may be used within the scope of embodiments of the present invention.

As indicated, in one embodiment, the ECG device 105 can be operated by emergency response and/or emergency room medical providers to perform the electrocardiogram procedure on the patient. For example, emergency response and/or emergency room medical providers can attach a number of electrodes on the patient's body to measure the electrical currents generated by the patient's heart. The ECG device 105 can then output/generate electrocardiographic information representative of the electrical currents generated by the patient's heart. The output/generated electrocardiographic information may be, for example, a raw ECG file. The raw ECG file may include 3-lead electrocardiographic traces/waveforms, 5-lead electrocardiographic traces/waveforms, 12-lead electrocardiographic traces/waveforms, and/or the like. Illustrative traces/waveforms are shown in FIG. 6. In one embodiment, depending on the electrical currents measured by the ECG device 105, the ECG device 105 may automatically classify the patient as STEMI. In another embodiment, the ECG device 105 may receive the appropriate classification from an emergency response and/or emergency room medical provider. As will be recognized, in other embodiments, a variety of tests, procedures, and/or the like may be performed on the patient to assist in evaluating and diagnosing a variety of medical conditions.

Because the patient has been classified as or is suspected of suffering from a STEMI, it may be important for medical providers with special training (e.g., specialty medical providers such as cardiologists) to timely review the electrocardiographic information output/generated by the ECG device 105. Specialty medical providers, for example, can use the electrocardiographic information to (a) diagnose, (b) confirm a diagnosis of, and/or (b) change a diagnosis of STEMI. Such accurate and prompt diagnosis may help, for example, (a) assure that proper care is administered to the patient and (b) limit false positive and/or false negative diagnoses. Thus, in one embodiment, to assist the appropriate medical providers in making diagnoses, the electrocardiographic information can be transmitted to the management system 100. As indicated, in one embodiment, the ECG device 105 may be configured to transmit the electrocardiographic information to the management system 100. In another embodiment, the electrocardiographic information is transmitted to the management system 100 with the assistance of another computing entity.

b. Receipt of Medical Information and Profiles

In one embodiment, as indicated in Block 400 of FIG. 4, the management system 100 (e.g., via the management module 250) can receive the electrocardiographic information (or medical information) transmitted from the ECG device 105 (or other computing entity). The electrocardiographic information may include, be associated with, and/or transmitted with patient information. For example, the patient information may include a variety of information, such as the patient's name, age, address, weight, birthday, medical record number, social security number, allergies, symptoms, medical conditions (e.g. hypoglycemia, hyperglycemia, etc.), and/or the like. In one embodiment, the management system 100 can use the patient information to identify and/or create a medical record associated with the patient (Block 405 of FIG. 4). The management system 100 can also electronically store the electrocardiographic information (or other medical information) in association with the medical record. (Block 410 of FIG. 4). For example, the electrocardiographic information, patient information, and/or medical record may be stored in a database 230. This information can be stored in the database 230 in a variety of formats, such as Health Level Seven International (“HL7”), Digital Imaging and Communications in Medicine (“DICOM”), and/or the like. Additionally or alternatively, the database 230 may be part of or in communication with a variety of computing entities, such as an electronic health records system. Such a configuration may allow the management system 100 to communicate the electrocardiographic information (or other medical information), patient information, and/or medical record to a variety of interested parties.

In one embodiment, in response to receiving the electrocardiographic information, the management system 100 can generate notifications indicating that a patient suspected of suffering a STEMI is en route to the health care facility or is within the health care facility. As part of this functionality, in one embodiment, the management system 100 can electronically store profiles that correspond respectively to medical providers. For example, a profile corresponding to a medical provider can be used to store attributes associated with the medical provider that may assist in providing (e.g., transmitting) the electrocardiographic information (or other medical information), patient information, and/or medical record to a mobile device 110, application, and/or account associated with the medical provider. The attributes stored in the profiles may include the respective medical provider's (a) name, (b) email addresses, (c) short message service (“SMS”) addresses, (d) phone numbers, (e) specialties, and/or (f) the like. As will be recognized, the profiles may also include a variety of other information and be customized to adapt to various needs.

In one embodiment, each medical provider profile (e.g., user) can also be associated with or assigned to one or more medical provider groups (e.g., user groups). The medical provider groups can be used, for example, to group medical providers (e.g., medical provider profiles) who (a) have similar specialties, (b) work at the same facility, (c) work in the same department within a facility, (d) work on the same team, and/or the like. For example, a medical provider group may comprise a team of medical providers who perform catheterization procedures at the health care facility or work in the cardiology department.

Thus, in one embodiment, via the profiles and in response to receiving the electrocardiographic information, the management system 100 can generate notifications that a patient suspected of suffering a STEMI is en route to the health care facility or is within the health care facility. For example, the management system 100 can generate notifications (e.g., via the provider alert module 270) to certain emergency room medical providers and on-call specialty medical providers (e.g., cardiologists) that a patient suspected of suffering a STEMI is en route to the health care facility or is within the health care facility. Such notifications can be in a variety of formats. For example, the notifications may include, for example, textual, graphical, and/or audible notifications, such as a pop-up notification being displayed via a user computing device 115. In one embodiment, the management system 100 can also monitor the notifications to determine if and when they were received, read, and/or heard. In various embodiments, these notifications may provide medical providers with advance notice and preparation time before being required to attend to the patient. As will be recognized a variety of approaches and techniques can be used to generate notifications to certain medical providers under various circumstances.

c. Transmission of Medical Information to Medical Providers

In one embodiment, the management system 100 can transmit at least a portion of the electrocardiographic information (or other medical information), patient information, and/or medical record to various computing entities. For example, in one embodiment using the above-discussed profiles, for example, the management system 100 can transmit the electrocardiographic information to a user computing device 115, account, and/or application associated with the health care facility to which the patient is being transported or at which the patient is being treated. For instance, the electrocardiographic information may be transmitted to a user computing device 115 via which an emergency room medical provider can view and access the electrocardiographic information (e.g., via the management module 250). This may allow the emergency room medical provider the ability to preliminarily diagnose the patient's medical condition. After an appropriate review by the emergency room medical provider, for example, it may be determined that it is necessary to consult a specialty medical provider (e.g., a cardiologist) to confirm the diagnosis. For instance, the emergency room medical provider may consider it necessary to consult a specialty medical provider (e.g., a cardiologist) to confirm that the patient is actually suffering from a STEMI. Thus, in one embodiment, the management system 100 can be used to transmit the electrocardiographic information to a mobile device 110, account, and/or application of a particular medical provider, such as an on-call cardiologist.

In one embodiment, using a profile associated with a specialty medical provider, the management system 100 can transmit the electrocardiographic information to a mobile device 110, account, and/or application associated with the specialty medical provider (Block 415 of FIG. 4). In one embodiment, the electrocardiographic information may be automatically pushed out to a mobile device 110, account, and/or application associated with the specialty medical provider from the management system 100. In another embodiment, the emergency room medical provider (e.g., operating a user computing device 115) can elect to transmit the electrocardiographic information to the mobile device 110, account, and/or application associated with the specialty medical provider from the management system 100.

In one embodiment, when the management system 100 transmits the electrocardiographic information to the mobile device 110, account, and/or application, the electrocardiographic information may be in a variety of formats. For example, in one embodiment, the electrocardiographic information may be a raw ECG file. In another embodiment, the electrocardiographic information can be in an encapsulated format or as an attachment using a variety of image and/or document formats. Depending on the format, the mobile device 110 receiving the electrocardiographic information, for example, may require an application to view the information (e.g., via the provider alert module 270).

In one embodiment, the management system 100 can filter the electrocardiographic information for compliance with applicable regulations (e.g., Health Insurance Portability and Accountability Act) prior to transmission to the mobile device 110. For example, in one embodiment, the management system 100 can anonymize the electrocardiographic information prior to transmission to the mobile device 110.

d. Transmission of Medical Information to Medical Providers

In one embodiment, in response to the management system 100 transmitting the electrocardiographic information to the mobile device 110, the mobile device 110 receives the electrocardiographic information (Block 500 of FIG. 5). The mobile device 110 can then display the electrocardiographic information for viewing by the specialty medical provider. Via input from the specialty medical provider, the mobile device 110 can zoom in, zoom out, and/or scroll through the electrocardiographic information. Moreover, the mobile device 110 can be used to change the lead display to facilitate review of the ECG waveforms/traces. As shown in FIG. 7, the mobile device 110 can also display the electrocardiographic information along with, for example, patient information and/or other relevant information. In various embodiments, this may allow the specialty medical provider to diagnose the patient's medical condition while being remote from the patient and/or health care facility. As will be recognized a variety of approaches and techniques can be used to view the electrocardiographic information (and/or other medical information).

In one embodiment, after reviewing the electrocardiographic information (or other medical information), the specialty medical provider can diagnose the patient (e.g., determine a patient status for the patient) based at least in part on, for example, the electrocardiographic information. The specialty medical provider can then input the patient status into the mobile device 110. This input can be entered into the mobile device 110 in a variety of ways. For example, as shown in FIG. 7, an application or program running on the mobile device 110 may cause display of the electrocardiographic information along with potential selections corresponding to different diagnoses. In other embodiments, the patient status can be input into the mobile device 110 via radio buttons, drop down boxes, textual input, voice recognition technologies, and/or a variety of other approaches and techniques. As will also be recognized, a variety of other approaches and techniques can be used.

In one embodiment, as indicated in Block 505, the mobile device 110 receives the patient status as input from the specialty medical provider. Continuing with the above example, the specialty medical provider can diagnose the patient as STEMI or non-STEMI and input this patient status by selecting the appropriate icon displayed by the mobile device 110. As indicated in Block 510, the mobile device 110 can then transmit the patient status to the management system 100 (Block 510 of FIG. 5).

e. Lab Alert and Protocol Initiation

As indicated, in one embodiment, the management system 100 can receive the patient status transmitted from the mobile device 110 (Block 420 of FIG. 4). In response to the management system 100 receiving the patient status, if appropriate, the management system 100 can automatically initiate one or more protocols associated with the patient status (Block 425 of FIG. 4). In one embodiment, a protocol may involve generating and transmitting notifications to certain medical provider groups (e.g., user groups) via profiles (e.g., via the lab alert module 260). For instance, a user group may include a group of medical providers who perform certain procedures at the health care facility or work in specific department. Continuing with the above example, a group of on-call medical providers assigned to perform catheterization procedures during a certain timeframe may be notified by the management system 100.

In one embodiment, the management system 100 can transmit the notifications to the medical providers' mobile phones, applications, and/or accounts. The notifications may include a variety of information, such as the patient's name and patient status. The management system 100 can also receive acknowledgements that the notified medical providers have received, viewed, and/or heard the notifications. Moreover, in one embodiment, the management system 100 may also receive responses from the notified medical providers with regard to their availability to assist in attending to the patient (e.g., participating in a catheterization procedure) and an estimated time of arrival at the health care facility. For example, a response (e.g., from a user of a user group) may indicate that the notified medical provider is en route to the health care facility or already at the health care facility. In one embodiment, the management system 100 can provide access to this information to a variety of interested parties. For example, in one embodiment, an emergency room medical provider can view the information and/or be notified that a cardiologist is en route to the health care facility to attend to the patient (e.g., to perform a catheterization procedure).

In one embodiment, the management system 100 can monitor and track all outbound notifications. The management system 100 can also track who has responded and who has yet to respond. In various embodiments, this may allow the management system 100 to generate and transmit additional notifications to other medical providers if no response has been received from the notified medical providers within a predetermined period of time. Continuing with the above example, the management system 100 can monitor the responses from the group of on-call medical providers (e.g., notified medical providers) assigned to perform catheterization procedures to determine if additional medical providers will be needed. In various embodiments, such notifications can also allow, for example, a group of medical providers who perform catheterization procedures to (a) travel to the health care facility, (b) prepare equipment and rooms to perform a procedure on a patient, (c) have advance notice of a patient's arrival, and/or (d) the like. In various embodiments, some of these concepts can also be used to track response times to certain medical conditions, such as door-to-balloon times for STEMI patients, to evaluate a health care facility's effectiveness in treating certain medical conditions.

In one embodiment, a protocol may also include a standard operating procedure that should be followed by medical providers and/or a health care facility when treating a patient with a certain medical condition (e.g., STEMI). For example, the protocol may include a checklist that should be completed within a predetermined period of time. An illustrative checklist for a STEMI patient may include alerting appropriate medical providers, reserving and preparing an operating room, receiving commitments that medical providers can start the procedures within 90 minutes, and/or the like.

In one embodiment, the management system 100 can provide or provide access to this type of information to a variety of users in many ways. For example, the management system 100 can allow this information to be viewed and accessed via a portal, browser, or other viewing application on a user computing device 115. Similarly, a browser can be embedded or opened in another application. As will be recognized, a variety of other approaches and techniques can be used.

f. Forwarding of Medical Information

In one embodiment, certain health care facilities may not be equipped to handle every medical condition. For example, not all health care facilities have equipment and/or medical providers who are capable of performing catheterization procedures. Thus, in one embodiment, the management system 100 can transmit the medical information (e.g., electrocardiographic information) to a health care facility to which the patient is being or will be transported (e.g., via the forward module 240). For example, the management system 100 can transmit the medical information to a computing entity at another health care facility and audit the transmission. The management system 100 can also transmit, for example, any known patient demographics, medical conditions, and other relevant information. The management system 100 can transmit the medical information (and/or other information) in a variety of formats, such as such as HL7, DICOM, and/or the like. As will be recognized a variety of other approaches and techniques may also be used.

### IV. Conclusion

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for managing electrocardiographic information, the method comprising:
receiving electrocardiographic information associated with a patient;
electronically identifying, via one or more processors, a provider associated with a health care facility;
transmitting at least a portion of the electrocardiographic information to a mobile device associated with the provider;
in response to transmitting at least a portion of the electrocardiographic information to the mobile device associated with the provider, receiving a patient status from the mobile device, wherein the patient status (a) is determined by the provider based at least in part on the at least a portion the electrocardiographic information and (b) is input by the provider via the mobile device; and
in response to receiving the patient status from the mobile device, automatically initiating a protocol associated with the patient status.

2. The method of claim 1, wherein (a) automatically initiating the protocol comprises generating one or more notifications to users of a user group and (b) the one or more notifications comprise the patient status.

3. The method of claim 2 further comprising, in response to generating the one or more notifications, receiving at least one response from a user of the user group.

4. The method of claim 1, wherein the patient status is selected from the group consisting of ST segment elevation myocardial infarction (STEMI) and non-ST segment elevation myocardial infarction (non-STEMI).

5. The method of claim 4, wherein (a) the received electrocardiographic information is transmitted from an electrocardiographic device and (b) the electrocardiographic device is located remote from the health care facility.

6. The method of claim 5, wherein (a) the received electrocardiographic information is transmitted from an electrocardiographic device and (b) the electrocardiographic device is located within the health care facility.

7. The method of claim 1 further comprising filtering the electrocardiographic information for compliance with applicable regulations prior to transmission of the at least a portion the electrocardiographic information to the mobile device.

8. A computer program product for managing electrocardiographic information, the computer program product comprising at least one computer-readable storage medium having computer-readable program code portions stored therein, the computer-readable program code portions comprising:
an executable code portion configured to receive electrocardiographic information associated with a patient;
an executable code portion configured to identify a provider associated with a health care facility;
an executable code portion configured to transmit at least a portion of the electrocardiographic information to a mobile device associated with the provider;
an executable code portion configured to, in response to transmitting at least a portion of the electrocardiographic information to the mobile device associated with the provider, receive a patient status from the mobile device, wherein the patient status (a) is determined by the provider based at least in part on the at least a portion the electrocardiographic information and (b) is input by the provider via the mobile device; and
an executable code portion configured to, in response to receiving the patient status from the mobile device, automatically initiate a protocol associated with the patient status.

9. The computer program product of claim 8, wherein (a) the executable code portion configured to automatically initiate the protocol is further configured to generate one or more notifications to users of a user group and (b) the one or more notifications comprise the patient status.

10. The computer program product of claim 9 further comprising an executable code portion configured to, in response to generating the one or more notifications, receive at least one response from a user of the user group.

11. The computer program product of claim 8, wherein the patient status is selected from the group consisting of ST segment elevation myocardial infarction (STEMI) and non-ST segment elevation myocardial infarction (non-STEMI).

12. The computer program product of claim 8, wherein (a) the received electrocardiographic information is transmitted from an electrocardiographic device and (b) the electrocardiographic device is located remote from the health care facility.

13. The computer program product of claim 12, wherein (a) the received electrocardiographic information is transmitted from an electrocardiographic device and (b) the electrocardiographic device is located within the health care facility.

14. The computer program product of claim 8 further comprising an executable code portion configured to filter the electrocardiographic information for compliance with applicable regulations prior to transmission of the at least a portion the electrocardiographic information to the mobile device.

15. An apparatus comprising at least one processor and at least one memory including computer program code, the at least one memory and the computer program code configured to, with the processor, cause the apparatus to at least:
receive electrocardiographic information associated with a patient;
identify a provider associated with a health care facility;
transmit at least a portion of the electrocardiographic information to a mobile device associated with the provider;
in response to transmitting at least a portion of the electrocardiographic information to the mobile device associated with the provider, receive a patient status from the mobile device, wherein the patient status (a) is determined by the provider based at least in part on the at least a portion the electrocardiographic information and (b) is input by the provider via the mobile device; and
in response to receiving the patient status from the mobile device, automatically initiate a protocol associated with the patient status.

16. The apparatus of claim 15, wherein (a) the memory and computer program code are further configured to, with the processor, cause the apparatus to generate one or more notifications to users of a user group and (b) the one or more notifications comprise the patient status.

17. The apparatus of claim 16, wherein the memory and computer program code are further configured to, with the processor, cause the apparatus to, in response to generating the one or more notifications, receive at least one response from a user of the user group.

18. The apparatus of claim 15, wherein the patient status is selected from the group consisting of ST segment elevation myocardial infarction (STEMI) and non-ST segment elevation myocardial infarction (non-STEMI).

19. The apparatus of claim 18, wherein the memory and computer program code are further configured to, with the processor, cause the apparatus to filter the electrocardiographic information for compliance with applicable regulations prior to transmission of the at least a portion the electrocardiographic information to the mobile device.
